(19) United States
(12) Patent Application Publication
(10) Pub. No.: US 2026/0183477 A1
(43) Pub. Date: Jul. 2, 2026

(54) Automatic Feature Management for Ambulatory Infusion Systems

(71) Applicant: Tandem Diabetes Care, Inc., San Diego, CA (US)

(72) Inventor: Paul Harris, San Diego, CA (US)

(21) Appl. No.: 19/429,961

(22) Filed: Dec. 22, 2025

Related U.S. Application Data
(60) Provisional application No. 63/740,202, filed on Dec. 30, 2024.

(51) Int. Cl. A61M 5/142

(52) U.S. Cl. CPC A61M 5/142; A61M 2005/14208; A61M 2202/0007; A61M 2202/04; A61M 2205/276; A61M 2205/3303; A61M 2205/3327; A61M 2205/3592; A61M 2205/502; A61M 2230/005; A61M 2230/04; A61M 2230/201

(57) ABSTRACT

The present disclosure regards an ambulatory infusion system with automatic feature management capabilities. The ambulatory infusion system includes an ambulatory infusion pump, a memory, and a processor. The ambulatory infusion pump is configured to provide a medicament to a user and communicate with a mobile device, where the mobile device is configured to execute an infusion-management application. The memory stores an advanced infusion feature for the ambulatory infusion pump or infusion-management application, where the advanced infusion feature is disabled by default. The processor is configured to monitor a competency level of the user or a physiological parameter of the user. The processor is also configured to determine whether an enablement criterion is satisfied based on the competency level of the user or the physiological parameter of the user. Additionally, the processor is configured to enable the advanced infusion feature responsive to determining that the enablement criterion is satisfied.

### RELATED APPLICATIONS

The present application claims the benefit of U.S. Provisional App. No. 63/740,202 (filed Dec. 30, 2024), the entirety of which is incorporated herein by reference.

### TECHNICAL FIELD

The present disclosure relates, generally, to medical devices and, more specifically, to ambulatory infusion pumps for diabetes management.

### BACKGROUND

Ambulatory infusion pumps have significantly simplified medication management for people with chronic conditions such as diabetes. Rather than requiring regular injections, these devices allow users to administer medicament boluses at the press of a button. Many ambulatory infusion pumps also offer programmable delivery rates, diagnostic data collection, and integration with other sensors and monitors.

As this technology continues to develop, it yields additional features and added functionality. However, such developments run the risk of overly complicating ambulatory infusion systems, particularly for users new to the technology. Some new users already struggle to appreciate modern pump capabilities. Left unaddressed, this issue will only increase as ambulatory infusion technologies become increasingly sophisticated and complex.

### SUMMARY

The present disclosure seeks to streamline the onboarding process for ambulatory infusion systems without sacrificing the features or functionality thereof. This streamlining involves designating certain features as “advanced” and hiding or at least disabling them during default operation. In this manner, a new user will only need to learn a subset of the features available to their ambulatory infusion pump or mobile device.

Once the user is ready, such advanced features can be automatically enabled for them. Determining readiness can depend, for instance, on user competence with respect to the ambulatory infusion pump, an infusion-management application running on the mobile device, or a disease treated by the ambulatory infusion pump. Also, readiness determinations can depend on a physiological parameter of the user, which could indicate that the user is in need of the advanced feature. These determinations are discussed in more detail hereinbelow, as are particular examples of advanced infusion features.

### Exemplary embodiments include the following:

An Ambulatory Infusion System. The ambulatory infusion system includes an ambulatory infusion pump, a memory, and a processor. The ambulatory infusion pump includes a pump mechanism configured to provide a medicament to a user and an antenna configured to facilitate wireless communication with a mobile device. The mobile device is configured to execute an infusion-management application for managing the ambulatory infusion pump. The memory stores an advanced infusion feature for the ambulatory infusion pump or infusion-management application. The advanced infusion feature is disabled by default. The processor is configured to monitor a competency level of the user with respect to the ambulatory infusion pump, the infusion-management application, or a disease of the user treatable with the medicament or a physiological parameter of the user. The processor is also configured to determine whether an enablement criterion is satisfied based on the competency level of the user or the physiological parameter of the user. Additionally, the processor is configured to, responsive to determining that the enablement criterion is satisfied, enable the advanced infusion feature.

A Mobile Device. The mobile device includes an antenna, a memory, and a processor. The antenna is configured to facilitate wireless communication with an ambulatory infusion pump, where the ambulatory infusion pump includes a pump mechanism configured to provide a medicament to a user. The memory stores an infusion-management application for managing the ambulatory infusion pump, as well as an advanced infusion feature for the ambulatory infusion pump or infusion-management application. The advanced infusion feature is disabled by default. The processor is configured to execute the infusion-management application. The processor is also configured to monitor a competency level of the user with respect to the ambulatory infusion pump, the infusion-management application, or a disease of the user treatable with the medicament or a physiological parameter of the user. Additionally, the processor is configured to determine whether an enablement criterion is satisfied based on the competency level of the user or the physiological parameter of the user. Further, the processor is configured to, responsive to determining that the enablement criterion is satisfied, enable the advanced infusion feature.

A Computer-Implemented Method. The computer-implemented method is for automatic feature management in an ambulatory infusion system, and the method includes wirelessly connecting an ambulatory infusion pump and a mobile device. The ambulatory infusion pump includes a pump mechanism configured to provide a medicament to a user, and the mobile device is configured to execute an infusion-management application for managing the ambulatory infusion pump. The method also includes monitoring a competency level of the user with respect to the ambulatory infusion pump, the infusion-management application, or a disease of the user treatable with the medicament or a physiological parameter of the user. Additionally, the method includes determining whether an enablement criterion is satisfied based on the competency level of the user or the physiological parameter of the user. Further, the method includes, responsive to determining that the enablement criterion is satisfied, enabling an advanced infusion feature for the ambulatory infusion pump or infusion-management application, wherein the advanced infusion feature is disabled by default.

A Computer-Readable Medium. The computer-readable medium is non-transitory, and the medium stores instructions that, when executed by a processor of an electronic device, cause the electronic device to perform operations. The operations include communicatively connecting an ambulatory infusion pump and a mobile device. The ambulatory infusion pump includes a pump mechanism configured to provide a medicament to a user, and the mobile device is configured to execute an infusion-management application for managing the ambulatory infusion pump. The operations also include monitoring a competency level of the user with respect to the ambulatory infusion pump, the infusion-management application, or a disease of the user treatable with the medicament or a physiological parameter of the user. Additionally, the operations include determining whether an enablement criterion is satisfied based on the competency level of the user or the physiological parameter of the user. Further, the operations include, responsive to determining that the enablement criterion is satisfied, enabling an advanced infusion feature for the ambulatory infusion pump or infusion-management application, wherein the advanced infusion feature is disabled by default.

Based on the following Detailed Description, other configurations of the subject technology will be apparent to those skilled in the art. The Detailed Description describes various configurations of the subject technology, particularly with respect to illustrations thereof. Nonetheless, the subject technology is capable of other and different configurations, and its several details are capable of modification in various other respects—all without departing from the scope of the subject technology. In short, the Drawings and Detailed Description are presented for illustrative purposes and should not be construed as restricting the present disclosure.

### BRIEF DESCRIPTION OF THE DRAWINGS

For a better understanding of the present disclosure, reference should be made to the Detailed Description, below, in conjunction with the following drawings. Like reference numerals refer to corresponding parts throughout the figures and the description.

FIG. 1 illustrates an example ambulatory infusion system for medicament delivery, according to various aspects of the subject technology.

FIGS. 2A to 2J illustrate an example infusion-management application with advanced infusion features, according to various aspects of the subject technology.

FIG. 3 illustrates an example process for automatic feature management in an ambulatory infusion system, according to various aspects of the subject technology.

### DETAILED DESCRIPTION

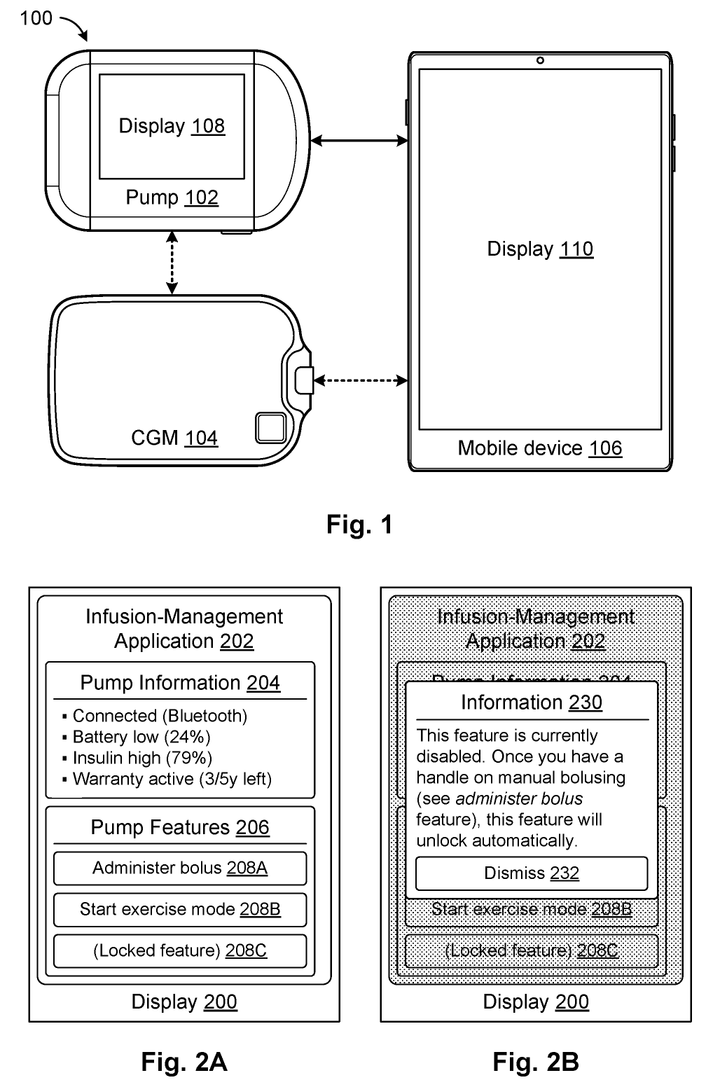

FIG. 1 illustrates an example ambulatory infusion system 100 for medicament delivery, according to various aspects of the subject technology. The system 100 includes an ambulatory infusion pump 102, a continuous glucose monitor 104 (CGM 104), and a mobile device 106. In the illustrated embodiment, both the pump 102 and mobile device 106 include respective displays 108 and 110; however, in some embodiments, the pump 102 does not include such a display 108 and instead communicates via other means such as the mobile device 102.

As illustrated, the pump 102 is configured to communicate with the mobile device 106 and, in some embodiments, with the CGM 104. Communications between the pump 102 and mobile device 106 can include the exchange of various data, instructions, and other information. The pump 102 is also configured to deliver a medicament to a user via a pumping mechanism, such as a peristaltic or syringe-based mechanism. Medicament delivery can include the delivery of a discrete bolus, or the delivery of the medicament at a particular flow rate (e.g., a basal rate).

The CGM 104 is configured to collect blood-glucose data indicative of a blood-glucose level of the user, which data is then transmitted by the CGM 104 to the pump 102 or the mobile device 106. This data can be used to determine when to administer a bolus of the medicament to the user or what flow rate to use for medicament delivery. In some embodiments, the pump 102 is configured to make these determinations itself after receiving the blood-glucose data, whether directly from the CGM 104 or via the mobile device 106. However, in some embodiments, the determinations are offloaded to the mobile device 106, which can then provide instructions to the pump 102 regarding medicament delivery.

Aside from potential delivery determinations, the mobile device 106 (e.g., a tablet, a smartphone, a smartwatch) is configured to communicate wirelessly with the ambulatory infusion pump 102. This communication can include the mobile device 106 forwarding blood-glucose data from the CGM 106 to the pump 102, sending medicament delivery instructions to the pump 102, or relaying physiological parameters or other information to the pump 102. Additionally, the communication can include the pump 102 sending notifications, operating data, or other information to the mobile device 106, which may be particularly useful in embodiments where the pump 102 lacks a display 108 and is therefore limited in communicating directly with the user.

Of particular note, one or both of the ambulatory infusion pump 102 and mobile device 106 include advanced infusion features that are disabled by default. With certain features disabled, the user is presented with only a subset of the total features of the ambulatory infusion pump 102 and mobile device 106. This can help streamline onboarding for new users, as the user will only need to familiarize themselves with non-disabled features. This can be especially helpful for users new to ambulatory infusion systems, such as the example system 100 of FIG. 1.

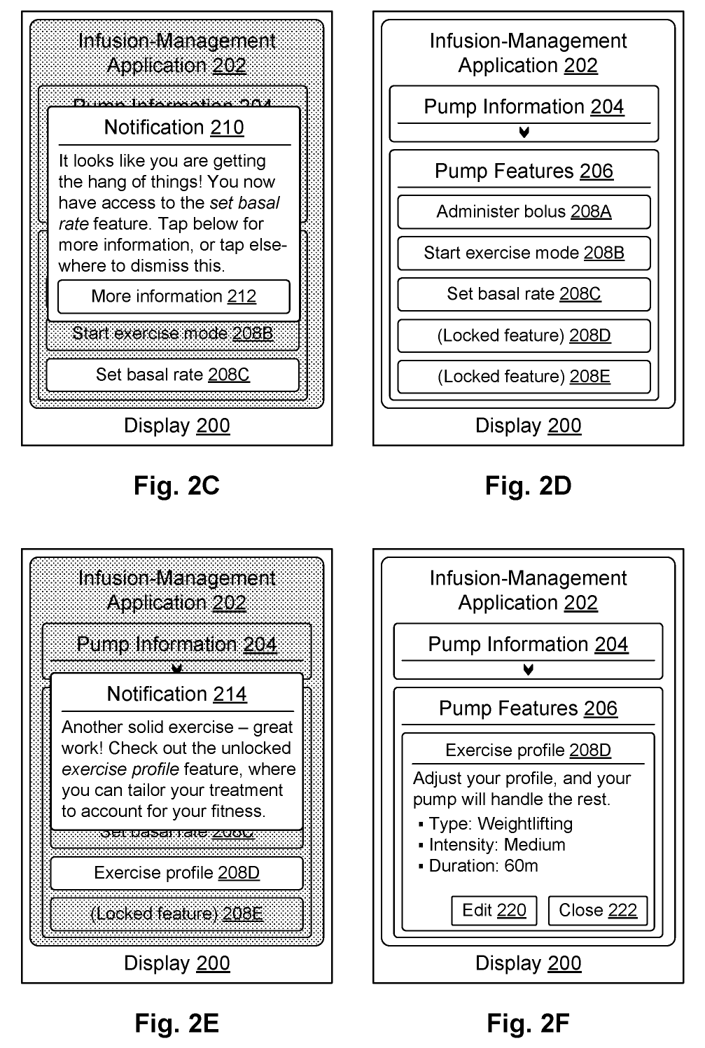

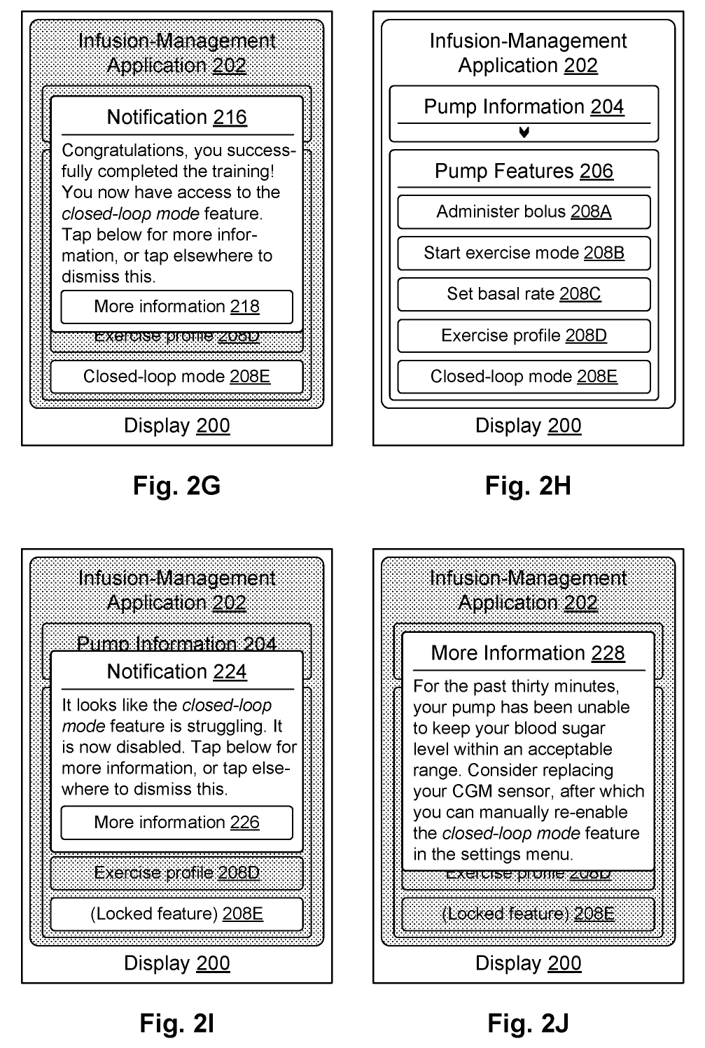

FIGS. 2A to 2J illustrate an example infusion-management application 202 with advanced infusion features 208C to 208E, according to various aspects of the subject technology. The infusion-management application 202 is displayed via a display 200 associated with an ambulatory infusion system (e.g., system 100). For example, the application 202 can be displayed at an ambulatory infusion pump (e.g., via display 108) or at a mobile device connected thereto (e.g., via display 110).

As illustrated in FIG. 2A, the application 202 includes information 204 regarding an ambulatory infusion pump (e.g., pump 102) of the aforenoted system. The information 204 indicates that the pump is currently connected, for instance, to a CGM (e.g., CGM 104) or a mobile device (e.g., device 106). The information 204 also indicates that the pump is low on battery, full of insulin, and still under warranty.

Additionally, the application 202 includes various features 206 for the application itself or for the ambulatory infusion pump. The first two of these features 206 are “basic” features that are enabled by default. They include an administer bolus feature 208A for requesting a bolus of medicament via the pump, and a start exercise mode feature 208B for initiating an exercise mode. The exercise mode can impact medicament delivery, for instance, by adjusting parameters (e.g., correction factor, basal rate) used in delivery determinations.

The third of the features 206 is an advanced feature 208C that is “locked” or disabled by default. By disabling advanced features, the infusion-management application 202 simplifies the onboarding process for the user, directing their energy towards the already enabled basic features. In some embodiments, the application 202 further streamlines onboarding by hiding disabled features (e.g., feature 208C) until they are enabled.

As illustrated in FIG. 2B, the application 202 can provide the user with information 230 regarding disabled features (e.g., feature 208C), for example, in response to the user attempting to access such features. This information 230 may guide the user towards other, already enabled features (e.g., feature 208A) and may also provide guidance on unlocking disabled features. A dismiss button 232 allows the user to dismiss the information 230.

For instance, the example application 202 enables by default the aforenoted administer bolus feature 208A. This feature 208A provides a close analog to self-administered injections, which users new to ambulatory infusion systems may already be familiar with. After the user is comfortable with the administer bolus feature 208A, the application 202 enables an advanced feature, the set basal rate feature 208C. This is illustrated in FIG. 2C, which includes a notification 210 indicating that the user is becoming more familiar with the infusion-management application 202 and also that the set basal rate feature 208C is now enabled. Additionally, the notification 210 includes a more information button 212, which directs the user to information regarding the enabled feature 208C (e.g., instructions).

In some embodiments, determining whether to enable a given feature involves monitoring a competency level of the user with respect to the application 202, the pump, or the user’s disease (e.g., diabetes). Competency can be determined, for instance, based on an amount of time since the user activated the ambulatory infusion pump, an amount of time since the user installed the infusion-management application on the mobile device, an amount of time since the user paired the ambulatory infusion pump with the mobile device, a use history of the ambulatory infusion pump or infusion-management application, an amount of time since a diagnosis of the user with the disease, an age of the user, or a prescription from a clinician of the user. In such embodiments, if the competency level of the user satisfies an enablement criterion for the advanced infusion feature, then the advanced infusion feature can be enabled.

FIG. 2D minimizes the pump information panel 204 to show more features 206, including disabled features 208D and 208E. The first of these disabled features, the exercise profile feature 208D, is enabled in FIG. 2E. As with the set basal rate feature 208C, the user receives a notification 214 when the exercise profile feature 208D is enabled, commending the user for exercising and directing them towards the exercise profile feature 208D.

Like the set basal rate feature 208C, the exercise profile feature 208D can be enabled based on user competency. For instance, if the application 202 determines that the user is sufficiently familiar with the related start exercise mode feature 208B (e.g., based on number of uses), the application 202 may then enable the exercise profile feature 208D. Alternatively, this feature 208D can be enabled based on a determination that the user exercises frequently and would therefore benefit from the feature 208D. Such a determination can be made based on a number of activations of the start exercise mode feature 208B; however, it can also depend on physiological parameters (e.g., heart rate) indicative of user exercise.

More generally, in some embodiments, determining whether to enable a given feature involves monitoring a physiological parameter of the user. This may include monitoring a heart rate of the user, for instance, to detect user exercise. Other potential physiological parameters for monitoring include a blood glucose level of the user, a hemoglobin A1C of the user, an insulin sensitivity of the user, a resting heart rate of the user, a blood pressure of the user, and a body mass index of the user. In such embodiments, if the physiological parameter of the user satisfies an enablement criterion for the advanced infusion feature, then the advanced infusion feature can be enabled.

In FIG. 2F, the exercise profile feature 208D is expanded to show various parameters that the user can modify to better tailor their exercise profile to their exercise regimen. The feature 208D includes a button 220 for editing the profile, as well as a button 222 for closing it. The parameters of the exercise profile are used to adjust medicament delivery determinations while the aforenoted exercise mode (see feature 208B) is active. In this manner, the advanced exercise profile feature 208D is integrated with the basic start exercise mode feature 208B. The two features 208B and 208D share a common goal—namely, tailoring medicament delivery to account for user exercise. The former feature 208B is sufficient for users that exercise infrequently, whereas the latter feature 208D offers improved functionality for users that exercise regularly or at high intensity. In this manner, advanced infusion features not only help to streamline onboarding but also serve to ensure the user has access to features they can most benefit from.

The final advanced feature, the closed-loop mode feature 208E, is enabled in FIG. 2G. A notification 216 congratulates the user on completing a training for the feature 208E and informs them regarding its enablement. The training can, for instance, instruct the user regarding the benefits and risks associated with closed-loop operation. Thus, training completion can also inform the user competency determinations discussed above. If the user completes the training or otherwise demonstrates competence sufficient for enablement of the feature 208E, the application can then enable it. As with some of the other notifications (e.g., notification 210), this notification 216 includes a button 218 for more information regarding the feature 208E.

In FIG. 2H, the infusion-management application 202 displays each of the pump features 206, including the enabled advanced features 208C to 208E. Then, in FIG. 2G, the application 202 displays a notification 224 indicating that the closed-loop feature 208E is struggling and is therefore disabled. This notification 224 includes a more information button 226, which leads to more information 228, displayed in FIG. 2J, regarding why the feature 208E was disabled and how the user can manually reenable it.

In some embodiments, an advanced feature is disabled if the feature has a negative impact on the user experience with the application 202 or on the user treatment via the ambulatory infusion pump. For instance, if the user is not using an enabled advanced feature, the application 202 can disable the feature to declutter its interface. As another example, if the feature is negatively impacting the blood-glucose level of the user, the application 202 can likewise disable the feature. Such is the case for the closed-loop feature 208E, which has been unable to keep the blood-glucose level of the user within an acceptable range. This could be due to faulty CGM measurements or an issue with the ambulatory infusion pump.

It is noted that many of the advanced features discussed herein could, in some embodiments, be basic features enabled by default and vice versa. Infusion features are not inherently basic or advanced; rather, the designer of the application 202, a designer of the pump, or a clinician of the user can determine which features should be enabled by default and which features should not. Similarly, the application 202 or pump can itself determine whether a feature should be enabled by default. For instance, this determination can occur when the application or pump first receives information regarding the user (e.g., indicative of whether the user will struggle with or have need for certain features).

Similarly, the various criteria for enabling the advanced infusion features are not set in stone. While criteria can be predetermined by the designer of the application or the designer of the pump, they can also be determined on a user-by-user basis, for instance, by the clinician of the user or by the application 202 or pump. For instance, this determination can occur when the application or pump first receives information regarding the user (e.g., indicative of circumstances that ought to occur prior to enabling certain features).

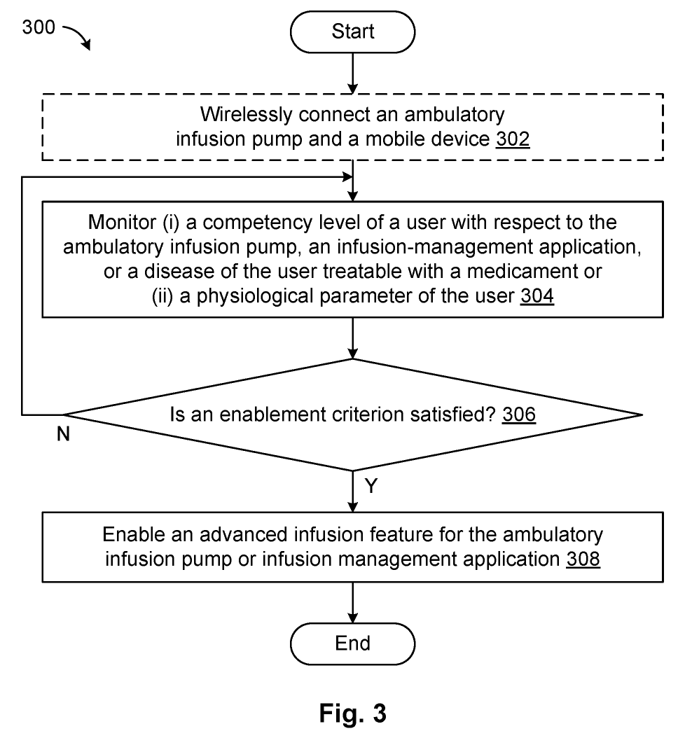

FIG. 3 illustrates an example process 300 for automatic feature management in an ambulatory infusion system (e.g., infusion system 100), according to various aspects of the subject technology. Operations of the process 300 can be executed by devices discussed above with respect to FIGS. 1 to 2J, such as the ambulatory infusion pump 102 or mobile device 106 of FIG. 1. Likewise, a server connected to such devices 102 or 106 could also execute one or more of the operations of the process 300.

In some embodiments, instructions stored in a non-transitory, computer-readable medium (e.g., memory of pump 102, or device 106) correspond to operations of the example process 300. In such embodiments, a processor of an electronic device (e.g., pump 102, device 106) can execute the instructions in order to cause the electronic device to perform the corresponding operations.

The process 300 optionally includes wirelessly connecting (302) an ambulatory infusion pump (e.g., pump 102) and a mobile device (e.g., device 106). The ambulatory infusion pump includes a pump mechanism configured to provide a medicament (e.g., insulin, glucagon) to a user, and the mobile device is configured to execute an infusion-management application (e.g., application 202) for managing the ambulatory infusion pump. As an example, connecting the ambulatory infusion pump and mobile device can be accomplished via Bluetooth, Wi-Fi, or other such wireless communication standards.

The process 300 also includes monitoring (304) a competency level of the user with respect to the ambulatory infusion pump, the infusion-management application, or a disease (e.g., diabetes) of the user treatable with the medicament or a physiological parameter of the user. Additionally, the process 300 includes determining whether an enablement criterion is satisfied based on the competency level of the user or the physiological parameter of the user. For example, this can include monitoring various parameters indicative of the competence level of the user, such as the amount of time the user has used the ambulatory infusion pump or infusion-management application, the amount of time since the user was diagnosed with the disease, and so on. As another example, this monitoring can include determining the competence level of the user based on one or more parameters indicative of said competence level. As yet another example, monitoring the physiological parameter can include monitoring a blood-glucose level of the user (e.g., collected by CGM 104) or a heart rate of the user (e.g., collected by mobile device 106 or a smartwatch connected thereto).

If the enablement criterion is not satisfied, then the process 300 returns to monitoring (304) the competency level of the user or the physiological parameter of the user. However, if the enablement criterion is satisfied, then the process 300 includes, responsive to determining that the enablement criterion is satisfied, enabling an advanced infusion feature (e.g., feature 208C, 208D, or 208E) for the ambulatory infusion pump or infusion-management application, where the advanced infusion feature is disabled by default. For example, the enablement criterion may require the competency level of the user satisfy (e.g., meet, exceed) a competency threshold, it may require the physiological parameter of the user satisfy a parameter threshold, or it may require some combination of the two.

In some embodiments, the process 300 further includes receiving a request for a use of the advanced infusion feature by the ambulatory infusion pump or infusion-management application and, responsive to receiving the request, determining whether the advanced infusion feature is enabled. If the advanced infusion feature is enabled, the process 300 further includes, responsive to determining that the advanced infusion feature is enabled, permitting the use of the advanced infusion feature. However, if the advanced infusion feature is not enabled, the process 300 further includes, responsive to determining that the advanced infusion feature is disabled, denying the use of the advanced infusion feature (see, e.g., information 230). For example, if the user requests access to a portion (e.g., exercise profile 208D) of the infusion-management application associated with the advanced feature (e.g., feature 208D), the user may be allowed to access said portion only if the corresponding advanced feature is enabled. As another example, if the user attempts to activate some function of the ambulatory infusion pump associated with the advanced feature (e.g., feature 208E), the function may activate only if the corresponding advanced feature is enabled.

In some embodiments, the advanced infusion feature is hidden by default from a user interface of the ambulatory infusion pump or infusion-management application. In such embodiments, the process 300 further includes, responsive to determining that the enablement criterion is satisfied, presenting an indication (e.g., notification 210, 214, or 216) of the advanced infusion feature via the user interface. For example, presenting the indication of the advanced infusion feature can include providing a notification to the user or simply displaying an option for the user to activate the advanced infusion feature (see, e.g., features 206).

In some embodiments, the process 300 further includes, responsive to determining that the enablement criterion is satisfied, providing a notification (e.g., notification 210, 214, or 216) to the user via the user interface of the ambulatory infusion pump or infusion-management application. In such embodiments, the notification indicates the advanced infusion feature is enabled and directs the user to instructions for using the advanced infusion feature (e.g., via button 212 or 218).

In some embodiments, the process 300 further includes, after enabling the advanced infusion feature, monitoring interactions of the user with the advanced infusion feature and a blood-sugar level of the user (e.g., collected by CGM 104). In such embodiments, the process 300 also includes determining whether the advanced infusion feature is negatively impacting the blood-sugar level of the user based on the interactions of the user and the blood-sugar level of the user. Additionally, the process 300 includes, responsive to determining that the advanced infusion feature is negatively impacting the blood-sugar level of the user, disabling the advanced infusion feature (e.g., feature 208E) and providing a notification (e.g., notification 224) to the user via a user interface of the ambulatory infusion pump or infusion-management application, where the notification indicates the advanced infusion feature is disabled and directs the user to instructions for re-enabling the advanced infusion feature (e.g., more information 228). For example, a closed-loop mode feature (e.g., feature 208E) can be disabled if it is negatively impacting the blood-sugar level of the user.

In some embodiments, the process 300 further includes receiving timing of exercises performed by the user via a first basic infusion feature (e.g., feature 208B) that is enabled by default and, responsive to receiving the timing of the exercises, adjusting operation of the ambulatory infusion pump based on the timing of the exercises. After enabling the advanced infusion feature, the process 300 further includes receiving a type or intensity of the exercises via the advanced infusion feature (e.g., feature 208D) and, responsive to receiving the type or intensity of the exercises, adjusting operation of the ambulatory infusion pump based on the type or intensity of the exercises. In such embodiments, determining that the enablement criterion is satisfied is based on the competency level of the user and includes determining that a use frequency of the first basic infusion feature satisfies a use-frequency threshold of the enablement criterion. For example, the enablement criterion may require that the user use the first basic infusion feature five, ten, or fifteen times before enabling the advanced infusion feature.

In some embodiments, the process 300 further includes receiving a request for a bolus of the medicament via a second basic infusion feature (e.g., feature 208A) that is enabled by default and, responsive to receiving the request for the bolus, causing the ambulatory infusion pump to provide the bolus to the user. After enabling the advanced infusion feature, the process 300 also includes receiving an indication of a basal rate and, responsive to receiving the basal rate, causing the ambulatory infusion pump to provide the medicament to the user at the basal rate. In such embodiments, determining that the enablement criterion is satisfied is based on the competency level of the user and includes determining that a number of boluses requested by the user satisfies a bolus-request threshold of the enablement criterion. For example, the enablement criterion may require that the user request ten, twenty, or fifty boluses before enabling the advanced infusion feature.

In some embodiments, the process 300 further includes determining the competency level of the user based on one or more of an amount of time since the user activated the ambulatory infusion pump, an amount of time since the user installed the infusion-management application on the mobile device, an amount of time since the user paired the ambulatory infusion pump with the mobile device, a use history of the ambulatory infusion pump or infusion-management application, an amount of time since a diagnosis of the user with the disease, an age of the user, and a prescription from a clinician of the user. In such embodiments, determining whether the enablement criterion is satisfied is based on the competency level of the user. For example, the enablement criterion may require that the user be sufficiently familiar with a particular feature (e.g., feature 208A or 208B) or with the infusion-management application itself (e.g., based on when the application was installed). As another example, the enablement criterion may require that the user be sufficiently familiar with ambulatory infusion systems (e.g., based on when the user activated the ambulatory infusion pump) or with the disease itself (e.g., based on when the user was diagnosed).

In some embodiments, the physiological parameter of the user includes one or more of a blood glucose level of the user, a hemoglobin A1C of the user, an insulin sensitivity of the user, a heart rate of the user, a blood pressure of the user, and a body mass index of the user. In such embodiments, determining whether the enablement criterion is satisfied is based on the physiological parameter of the user. For example, the enablement criterion may require that the user exercise a certain number of times each week, which can be determined based on the heart rate of the user. As another example, the enablement criterion may require that the user be a high-risk diabetic, which can be determined based on the hemoglobin A1C of the user.

In some embodiments, the process 300 further includes monitoring the competency level of the user or another physiological parameter of the user and determining whether another enablement criterion is satisfied based on the competency level or the other physiological parameter. If the other enablement criterion is not satisfied, then the process 300 returns to monitoring the competency level or the other physiological parameter. However, if the other enablement criterion is satisfied, then the process 300 includes, responsive to determining that the other enablement criterion is satisfied, enabling another advanced infusion feature (e.g., feature 208C, 208D, or 208E) for the ambulatory infusion pump or infusion-management application, where the other advanced infusion feature is disabled by default. The process 300 can also involve monitoring, determining, and enabling for more than two advanced infusion features, as discussed for FIGS. 2A to 2J.

Illustrative Clauses. For further reference, example aspects of the present disclosure are included below as numbered clauses. These clauses are provided for illustrative purposes and are not intended to limit the subject technology.

Clause 1. An ambulatory infusion system comprising: an ambulatory infusion pump that comprises (i) a pump mechanism configured to provide a medicament to a user and (ii) an antenna configured to facilitate wireless communication with a mobile device, wherein the mobile device is configured to execute an infusion-management application for managing the ambulatory infusion pump; a memory that stores an advanced infusion feature for the ambulatory infusion pump or infusion-management application, wherein the advanced infusion feature is disabled by default; a processor configured to: monitor (i) a competency level of the user with respect to the ambulatory infusion pump, the infusion-management application, or a disease of the user treatable with the medicament or (ii) a physiological parameter of the user; determine whether an enablement criterion is satisfied based on (i) the competency level of the user or (ii) the physiological parameter of the user; and responsive to determining that the enablement criterion is satisfied, enable the advanced infusion feature.

Clause 2. The ambulatory infusion system of clause 1, wherein the processor is further configured to: receive a request for a use of the advanced infusion feature by the ambulatory infusion pump or infusion-management application; responsive to receiving the request, determine whether the advanced infusion feature is enabled; responsive to determining that the advanced infusion feature is enabled, permit the use of the advanced infusion feature; and responsive to determining that the advanced infusion feature is disabled, deny the use of the advanced infusion feature.

Clause 3. The ambulatory infusion system of either clause 1 or 2, wherein: the advanced infusion feature is hidden by default from a user interface of the ambulatory infusion pump or infusion-management application; and the processor is further configured to, responsive to determining that the enablement criterion is satisfied, present an indication of the advanced infusion feature via the user interface.

Clause 4. The ambulatory infusion system of clause 3, wherein the processor is further configured to, responsive to determining that the enablement criterion is satisfied, provide a notification to the user via the user interface of the ambulatory infusion pump or infusion-management application, wherein the notification indicates the advanced infusion feature is enabled and directs the user to instructions for using the advanced infusion feature.

Clause 5. The ambulatory infusion system of any one of clauses 1 to 4, wherein the processor is further configured to, after enabling the advanced infusion feature: monitor (i) interactions of the user with the advanced infusion feature and (ii) a blood-sugar level of the user; determine whether the advanced infusion feature is negatively impacting the blood-sugar level of the user based on (i) the interactions of the user and (ii) the blood-sugar level of the user; and responsive to determining that the advanced infusion feature is negatively impacting the blood-sugar level of the user, disable the advanced infusion feature and provide a notification to the user via a user interface of the ambulatory infusion pump or infusion-management application, wherein

the notification indicates the advanced infusion feature is disabled and directs the user to instructions for re-enabling the advanced infusion feature.

Clause 6. The ambulatory infusion system of any one of clauses 1 to 5, wherein: the memory further stores a first basic infusion feature for the ambulatory infusion pump or infusion-management application, wherein the first basic infusion feature is (i) enabled by default and (ii) allows the user to indicate timing of exercises performed by the user, wherein the processor is further configured to adjust operation of the ambulatory infusion pump based on the timing of the exercises; the advanced infusion feature allows the user to indicate a type or intensity of the exercises, wherein the processor is further configured to adjust operation of the ambulatory infusion pump based on the type or intensity of the exercises; and determining that the enablement criterion is satisfied (i) is based on the competency level of the user and (ii) comprises determining that a use frequency of the first basic infusion feature satisfies a use-frequency threshold of the enablement criterion.

Clause 7. The ambulatory infusion system of any one of clauses 1 to 6, wherein: the memory further stores a second basic infusion feature for the ambulatory infusion pump or infusion-management application, wherein the second basic infusion feature is (i) enabled by default and (ii) allows the user to request a bolus of the medicament, wherein the processor is further configured to cause the ambulatory infusion pump to provide the bolus to the user responsive to the user requesting the bolus; the advanced infusion feature allows the user to indicate a basal rate for the medicament, wherein the processor is further configured to cause the ambulatory infusion pump to provide the medicament to the user at the basal rate responsive to the user indicating the basal rate; and determining that the enablement criterion is satisfied (i) is based on the competency level of the user and (ii) comprises determining that a number of boluses requested by the user satisfies a bolus-request threshold of the enablement criterion.

Clause 8. The ambulatory infusion system of any one of clauses 1 to 7, wherein: the processor is further configured to determine the competency level of the user based on two or more of (i) an amount of time since the user activated the ambulatory infusion pump, (ii) an amount of time since the user installed the infusion-management application on the mobile device, (iii) an amount of time since the user paired the ambulatory infusion pump with the mobile device, (iv) a use history of the ambulatory infusion pump or infusion-management application, (v) an amount of time since a diagnosis of the user with the disease, (vi) an age of the user, and (vii) a prescription from a clinician of the user; and determining whether the enablement criterion is satisfied is based on the competency level of the user.

Clause 9. The ambulatory infusion system of any one of clauses 1 to 8, wherein: the physiological parameter of the user comprises two or more of (i) a blood glucose level of the user, (ii) a hemoglobin A1C of the user, (iii) an insulin sensitivity of the user, (iv) a heart rate of the user, (v) a blood pressure of the user, and (vi) a body mass index of the user; and determining whether the enablement criterion is satisfied is based on the physiological parameter of the user.

Clause 10. The ambulatory infusion system of any one of clauses 1 to 9, wherein: the memory further stores instructions associated with another advanced infusion feature for the ambulatory infusion pump or infusion-management application, wherein the other advanced infusion feature is disabled by default; and the processor is further configured to: monitor (i) the competency level of the user or (ii) another physiological parameter of the user; determine whether another enablement criterion is satisfied based on (i) the competency level of the user or (ii) the other physiological parameter of the user; and responsive to determining that the other enablement criterion is satisfied, enable the other advanced infusion feature.

Clause 11. A mobile device comprising: an antenna configured to facilitate wireless communication with an ambulatory infusion pump, wherein the ambulatory infusion pump comprises a pump mechanism configured to provide a medicament to a user; a memory that stores (i) an infusion-management application for managing the ambulatory infusion pump and (ii) an advanced infusion feature for the ambulatory infusion pump or infusion-management application, wherein the advanced infusion feature is disabled by default and ; and a processor configured to: execute the infusion-management application; monitor (i) a competency level of the user with respect to the ambulatory infusion pump, the infusion-management application, or a disease of the user treatable with the medicament or (ii) a physiological parameter of the user; determine whether an enablement criterion is satisfied based on (i) the competency level of the user or (ii) the physiological parameter of the user; and responsive to determining that the enablement criterion is satisfied, enable the advanced infusion feature.

Clause 12. The mobile device of clause 11, wherein the processor is further configured to: receive a request for a use of the advanced infusion feature by the infusion-management application; responsive to receiving the request, determine whether the advanced infusion feature is enabled; responsive to determining that the advanced infusion feature is enabled, permit the use of the advanced infusion feature; and responsive to determining that the advanced infusion feature is disabled, deny the use of the advanced infusion feature.

Clause 13. The mobile device of either clause 11 or 12, wherein: the advanced infusion feature is hidden by default from a user interface of the infusion-management application; and the processor is further configured to, responsive to determining that the enablement criterion is satisfied, present an indication of the advanced infusion feature via the user interface.

Clause 14. The mobile device of clause 13, wherein the processor is further configured to, responsive to determining that the enablement criterion is satisfied, provide a notification to the user via the user interface of the infusion-management application, and the notification indicates the advanced infusion feature is enabled and directs the user to instructions for using the advanced infusion feature.

Clause 15. The mobile device of any one of clauses 11 to 14, wherein the processor is further configured to, after enabling the advanced infusion feature: monitor (i) interactions of the user with the advanced infusion feature and (ii) a blood-sugar level of the user; determine whether the advanced infusion feature is negatively impacting the blood-sugar level of the user based on (i) the interactions of the user and (ii) the blood-sugar level of the user; and responsive to determining that the advanced infusion feature is negatively impacting the blood-sugar level of the user, disable the advanced infusion feature and provide a notification to the user via a user interface of the infusion-management application, wherein the notification indicates the advanced infusion feature is disabled and directs the user to instructions for re-enabling the advanced infusion feature.

Clause 16. The mobile device of any one of clauses 11 to 15, wherein: the memory further stores a first basic infusion feature for the infusion-management application, wherein the first basic infusion feature is (i) enabled by default and (ii) allows the user to indicate timing of exercises performed by the user, wherein the processor is further configured to adjust operation of the ambulatory infusion pump based on the timing of the exercises; the advanced infusion feature allows the user to indicate a type or intensity of the exercises, wherein the processor is further configured to adjust operation of the ambulatory infusion pump based on the type or intensity of the exercises; and determining that the enablement criterion is satisfied (i) is based on the competency level of the user and (ii) comprises determining that a use frequency of the first basic infusion feature satisfies a use-frequency threshold of the enablement criterion.

Clause 17. The mobile device of any one of clauses 11 to 16, wherein: the memory further stores a second basic infusion feature for the ambulatory infusion pump or infusion-management application, wherein the second basic infusion feature is (i) enabled by default and (ii) allows the user to request a bolus of the medicament, wherein the processor is further configured to cause the ambulatory infusion pump to provide the bolus to the user responsive to the user requesting the bolus; the advanced infusion feature allows the user to indicate a basal rate for the medicament, wherein the processor is further configured to cause the ambulatory infusion pump to provide the medicament to the user at the basal rate responsive to the user indicating the basal rate; and determining that the enablement criterion is satisfied (i) is based on the competency level of the user and (ii) comprises determining that a number of boluses requested by the user satisfies a bolus-request threshold of the enablement criterion.

Clause 18. The mobile device of any one of clauses 11 to 17, wherein: the processor is further configured to determine the competency level of the user based on two or more of (i) an amount of time since the user activated the ambulatory infusion pump, (ii) an amount of time since the user installed the infusion-management application on the mobile device, (iii) an amount of time since the user paired the ambulatory infusion pump with the mobile device, (iv) a use history of the ambulatory infusion pump or infusion-management application, (v) an amount of time since a diagnosis of the user with the disease, (vi) an age of the user, and (vii) a prescription from a clinician of the user; and determining whether the enablement criterion is satisfied is based on the competency level of the user.

Clause 19. The mobile device of any one of clauses 11 to 18, wherein: the physiological parameter of the user comprises two or more of (i) a blood glucose level of the user, (ii) a hemoglobin A1C of the user, (iii) an insulin sensitivity of the user, (iv) a heart rate of the user, (v) a blood pressure of the user, and (vi) a body mass index of the user; and determining whether the enablement criterion is satisfied is based on the physiological parameter of the user.

Clause 20. The mobile device of any one of clauses 11 to 19, wherein: the memory further stores instructions associated with another advanced infusion feature for the infusion-management application, wherein the other advanced infusion feature is disabled by default; and the processor is further configured to: monitor (i) the competency level of the user or (ii) another physiological parameter of the user; determine whether another enablement criterion is satisfied based on (i) the competency level of the user or (ii) the other physiological parameter of the user; and responsive to determining that the other enablement criterion is satisfied, enable the other advanced infusion feature.

Clause 21. A computer-implemented method for automatic feature management in an ambulatory infusion system, the method comprising: wirelessly connecting an ambulatory infusion pump and a mobile device, wherein the ambulatory infusion pump comprises a pump mechanism configured to provide a medicament to a user, and the mobile device is configured to execute an infusion-management application for managing the ambulatory infusion pump; monitoring (i) a competency level of the user with respect to the ambulatory infusion pump, the infusion-management application, or a disease of the user treatable with the medicament or (ii) a physiological parameter of the user; determining whether an enablement criterion is satisfied based on (i) the competency level of the user or (ii) the physiological parameter of the user; and responsive to determining that the enablement criterion is satisfied, enabling an advanced infusion feature for the ambulatory infusion pump or infusion-management application, wherein the advanced infusion feature is disabled by default.

Clause 22. The computer-implemented method of clause 21, further comprising: receiving a request for a use of the advanced infusion feature by the ambulatory infusion pump or infusion-management application; responsive to receiving the request, determining whether the advanced infusion feature is enabled; responsive to determining that the advanced infusion feature is enabled, permitting the use of the advanced infusion feature; and responsive to determining that the advanced infusion feature is disabled, denying the use of the advanced infusion feature.

Clause 23. The computer-implemented method of either clause 21 or 22, wherein: the advanced infusion feature is hidden by default from a user interface of the ambulatory infusion pump or infusion-management application; and the method further comprises, responsive to determining that the enablement criterion is satisfied, presenting an indication of the advanced infusion feature via the user interface.

Clause 24. The computer-implemented method of clause 23, further comprising, responsive to determining that the enablement criterion is satisfied, providing a notification to the user via the user interface of the ambulatory infusion pump or infusion-management application, wherein the notification indicates the advanced infusion feature is enabled and directs the user to instructions for using the advanced infusion feature.

Clause 25. The computer-implemented method of any one of clauses 21 to 24, further comprising, after enabling the advanced infusion feature: monitoring (i) interactions of the user with the advanced infusion feature and (ii) a blood-sugar level of the user; determining whether the advanced infusion feature is negatively impacting the blood-sugar level of the user based on (i) the interactions of the user and (ii) the blood-sugar level of the user; and responsive to determining that the advanced infusion feature is negatively impacting the blood-sugar level of the user, disabling the advanced infusion feature and providing a notification to the user via a user interface of the ambulatory infusion pump or infusion-management application, wherein the notification indicates the advanced infusion feature is disabled and directs the user to instructions for re-enabling the advanced infusion feature.

Clause 26. The computer-implemented method of any one of clauses 21 to 25, further comprising: receiving timing of exercises performed by the user via a first basic infusion feature that is enabled by default; responsive to receiving the timing of the exercises, adjusting operation of the ambulatory infusion pump based on the timing of the exercises; after enabling the advanced infusion feature, receiving a type or intensity of the exercises via the advanced infusion feature; and responsive to receiving the type or intensity of the exercises, adjusting operation of the ambulatory infusion pump based on the type or intensity of the exercises; wherein determining that the enablement criterion is satisfied (i) is based on the competency level of the user and (ii) comprises determining that a use frequency of the first basic infusion feature satisfies a use-frequency threshold of the enablement criterion.

Clause 27. The computer-implemented method of any one of clauses 21 to 26, further comprising: receiving a request for a bolus of the medicament via a second basic infusion feature that is enabled by default; responsive to receiving the request for the bolus, causing the ambulatory infusion pump to provide the bolus to the user; after enabling the advanced infusion feature, receiving an indication of a basal rate; and responsive to receiving the basal rate, causing the ambulatory infusion pump to provide the medicament to the user at the basal rate; wherein determining that the enablement criterion is satisfied (i) is based on the competency level of the user and (ii) comprises determining that a number of boluses requested by the user satisfies a bolus-request threshold of the enablement criterion.

Clause 28. The computer-implemented method of any one of clauses 21 to 27, further comprising: determining the competency level of the user based on two or more of (i) an amount of time since the user activated the ambulatory infusion pump, (ii) an amount of time since the user installed the infusion-management application on the mobile device, (iii) an amount of time since the user paired the ambulatory infusion pump with the mobile device, (iv) a use history of the ambulatory infusion pump or infusion-management application, (v) an amount of time since a diagnosis of the user with the disease, (vi) an age of the user, and (vii) a prescription from a clinician of the user; wherein determining whether the enablement criterion is satisfied is based on the competency level of the user.

Clause 29. The computer-implemented method of any one of clauses 21 to 28, wherein: the physiological parameter of the user comprises two or more of (i) a blood glucose level of the user, (ii) a hemoglobin A1C of the user, (iii) an insulin sensitivity of the user, (iv) a heart rate of the user, (v) a blood pressure of the user, and (vi) a body mass index of the user; and determining whether the enablement criterion is satisfied is based on the physiological parameter of the user.

Clause 30. The computer-implemented method of any one of clauses 21 to 29, further comprising: monitoring (i) the competency level of the user or (ii) another physiological parameter of the user; determining whether another enablement criterion is satisfied based on (i) the competency level of the user or (ii) the other physiological parameter of the user; and responsive to determining that the other enablement criterion is satisfied, enabling another advanced infusion feature for the ambulatory infusion pump or infusion-management application, wherein the other advanced infusion feature is disabled by default.

Clause 31. A non-transitory, computer-readable medium storing instructions that, when executed by a processor of an electronic device, cause the electronic device to perform operations comprising: communicatively connecting an ambulatory infusion pump and a mobile device, wherein the ambulatory infusion pump comprises a pump mechanism configured to provide a medicament to a user, and the mobile device is configured to execute an infusion-management application for managing the ambulatory infusion pump; monitoring (i) a competency level of the user with respect to the ambulatory infusion pump, the infusion-management application, or a disease of the user treatable with the medicament or (ii) a physiological parameter of the user; determining whether an enablement criterion is satisfied based on (i) the competency level of the user or (ii) the physiological parameter of the user; and responsive to determining that the enablement criterion is satisfied, enabling an advanced infusion feature for the ambulatory infusion pump or infusion-management application, wherein the advanced infusion feature is disabled by default.

Clause 32. The non-transitory, computer-readable medium of clause 31, wherein the operations further comprise: receiving a request for a use of the advanced infusion feature by the ambulatory infusion pump or infusion-management application; responsive to receiving the request, determining whether the advanced infusion feature is enabled; responsive to determining that the advanced infusion feature is enabled, permitting the use of the advanced infusion feature; and responsive to determining that the advanced infusion feature is disabled, denying the use of the advanced infusion feature.

Clause 33. The non-transitory, computer-readable medium of either clause 31 or 32, wherein: the advanced infusion feature is hidden by default from a user interface of the ambulatory infusion pump or infusion-management application; and the operations further comprise, responsive to determining that the enablement criterion is satisfied, presenting an indication of the advanced infusion feature via the user interface.

Clause 34. The non-transitory, computer-readable medium of any one of clauses 31 to 33, wherein the operations further comprise, responsive to determining that the enablement criterion is satisfied, providing a notification to the user via the user interface of the ambulatory infusion pump or infusion-management application, and the notification indicates the advanced infusion feature is enabled and directs the user to instructions for using the advanced infusion feature.

Clause 35. The non-transitory, computer-readable medium of any one of clauses 31 to 34, wherein the operations further comprise, after enabling the advanced infusion feature: monitoring (i) interactions of the user with the advanced infusion feature and (ii) a blood-sugar level of the user; determining whether the advanced infusion feature is negatively impacting the blood-sugar level of the user based on (i) the interactions of the user and (ii) the blood-sugar level of the user; and responsive to determining that the advanced infusion feature is negatively impacting the blood-sugar level of the user, disabling the advanced infusion feature and providing a notification to the user via a user interface of the ambulatory infusion pump or infusion-management application, wherein the notification indicates the advanced infusion feature is disabled and directs the user to instructions for re-enabling the advanced infusion feature.

Clause 36. The non-transitory, computer-readable medium of any one of clauses 31 to 35, wherein the operations further comprise: receiving timing of exercises performed by the user via a basic infusion feature that is enabled by default; responsive to receiving the timing of the exercises, adjusting operation of the ambulatory infusion pump based on the timing of the exercises; after enabling the advanced infusion feature, receiving a type or intensity of the exercises via the advanced infusion feature; and responsive to receiving the type or intensity of the exercises, adjusting operation of the ambulatory infusion pump based on the type or intensity of the exercises; wherein determining that the enablement criterion is satisfied (i) is based on the competency level of the user and (ii) comprises determining that a use frequency of the basic infusion feature satisfies a use-frequency threshold of the enablement criterion.

Clause 37. The non-transitory, computer-readable medium of any one of clauses 31 to 36, wherein the operations further comprise: receiving a request for a bolus of the medicament via a basic infusion feature that is enabled by default; responsive to receiving the request for the bolus, causing the ambulatory infusion pump to provide the bolus to the user; after enabling the advanced infusion feature, receiving an indication of a basal rate; and responsive to receiving the basal rate, causing the ambulatory infusion pump to provide the medicament to the user at the basal rate; wherein determining that the enablement criterion is satisfied (i) is based on the competency level of the user and (ii) comprises determining that a number of boluses requested by the user satisfies a bolus-request threshold of the enablement criterion.

Clause 38. The non-transitory, computer-readable medium of any one of clauses 31 to 37, wherein: the operations further comprise determining the competency level of the user based on two or more of (i) an amount of time since the user activated the ambulatory infusion pump, (ii) an amount of time since the user installed the infusion-management application on the mobile device, (iii) an amount of time since the user paired the ambulatory infusion pump with the mobile device, (iv) a use history of the ambulatory infusion pump or infusion-management application, (v) an amount of time since a diagnosis of the user with the disease, (vi) an age of the user, and (vii) a prescription from a clinician of the user; and determining whether the enablement criterion is satisfied is based on the competency level of the user.

Clause 39. The non-transitory, computer-readable medium of any one of clauses 31 to 38, wherein: the physiological parameter of the user comprises two or more of (i) a blood glucose level of the user, (ii) a hemoglobin A1C of the user, (iii) an insulin sensitivity of the user, (iv) a heart rate of the user, (v) a blood pressure of the user, and (vi) a body mass index of the user; and determining whether the enablement criterion is satisfied is based on the physiological parameter of the user.

Clause 40. The non-transitory, computer-readable medium of any one of clauses 31 to 39, wherein the operations further comprise: monitoring (i) the competency level of the user or (ii) another physiological parameter of the user; determining whether another enablement criterion is satisfied based on (i) the competency level of the user or (ii) the other physiological parameter of the user; and responsive to determining that the other enablement criterion is satisfied, enabling another advanced infusion feature for the ambulatory infusion pump or infusion-management application, wherein the other advanced infusion feature is disabled by default.

Further Consideration. The specific order or hierarchy of steps in the processes disclosed herein is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Headings and subheadings, if any, are used for convenience only and do not limit the invention described herein.

The predicate words “configured to,” “operable to,” and “programmed to” do not imply any particular tangible or intangible modification of a subject, but rather are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as “implementations” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein, the terms “determine” and “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some embodiments, include a signal utilized to transmit one or more representations of the information. While recited in the singular, it will be appreciated that a message may be composed, transmitted, stored, received, and so on in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some embodiments, an n-input switch may be included to provide selective functionality where n is the number of inputs used to make the selection.

As used herein, the terms “correspond” or “corresponding” encompasses a structural, functional, quantitative, or qualitative correlation or relationship between two or more objects, data sets, information and the like, preferably where the correspondence or relationship may be used to translate one or more of the two or more objects, data sets, information and the like so to appear to be the same or equal. Correspondence may be assessed using one or more of a threshold, a value range, fuzzy logic, pattern matching, a machine-learning assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

What is claimed is:

1. An ambulatory infusion system comprising:
an ambulatory infusion pump that comprises (i) a pump mechanism configured to provide a medicament to a user and (ii) an antenna configured to facilitate wireless communication with a mobile device, wherein the mobile device is configured to execute an infusion-management application for managing the ambulatory infusion pump;
a memory that stores an advanced infusion feature for the ambulatory infusion pump or infusion-management application, wherein the advanced infusion feature is disabled by default;
a processor configured to:
monitor (i) a competency level of the user with respect to the ambulatory infusion pump, the infusion-management application, or a disease of the user treatable with the medicament or (ii) a physiological parameter of the user;
determine whether an enablement criterion is satisfied based on (i) the competency level of the user or (ii) the physiological parameter of the user; and
responsive to determining that the enablement criterion is satisfied, enable the advanced infusion feature.

2. The ambulatory infusion system of claim 1, wherein the processor is further configured to:
receive a request for a use of the advanced infusion feature by the ambulatory infusion pump or infusion-management application;
responsive to receiving the request, determine whether the advanced infusion feature is enabled;
responsive to determining that the advanced infusion feature is enabled, permit the use of the advanced infusion feature; and
responsive to determining that the advanced infusion feature is disabled, deny the use of the advanced infusion feature.

3. The ambulatory infusion system of claim 1, wherein:
the advanced infusion feature is hidden by default from a user interface of the ambulatory infusion pump or infusion-management application; and
the processor is further configured to, responsive to determining that the enablement criterion is satisfied, present an indication of the advanced infusion feature via the user interface.

4. The ambulatory infusion system of claim 3, wherein the processor is further configured to, responsive to determining that the enablement criterion is satisfied, provide a notification to the user via the user interface of the ambulatory infusion pump or infusion-management application, wherein the notification indicates the advanced infusion feature is enabled and directs the user to instructions for using the advanced infusion feature.

5. The ambulatory infusion system of claim 1, wherein the processor is further configured to, after enabling the advanced infusion feature:
monitor (i) interactions of the user with the advanced infusion feature and (ii) a blood-sugar level of the user;
determine whether the advanced infusion feature is negatively impacting the blood-sugar level of the user based on (i) the interactions of the user and (ii) the blood-sugar level of the user; and
responsive to determining that the advanced infusion feature is negatively impacting the blood-sugar level of the user, disable the advanced infusion feature and provide a notification to the user via a user interface of the ambulatory infusion pump or infusion-management application, wherein the notification indicates the advanced infusion feature is disabled and directs the user to instructions for re-enabling the advanced infusion feature.

6. The ambulatory infusion system of claim 1, wherein:
the memory further stores a first basic infusion feature for the ambulatory infusion pump or infusion-management application, wherein the first basic infusion feature is (i) enabled by default and (ii) allows the user to indicate timing of exercises performed by the user, wherein the processor is further configured to adjust operation of the ambulatory infusion pump based on the timing of the exercises;
the advanced infusion feature allows the user to indicate a type or intensity of the exercises, wherein the processor is further configured to adjust operation of the ambulatory infusion pump based on the type or intensity of the exercises; and
determining that the enablement criterion is satisfied (i) is based on the competency level of the user and (ii) comprises determining that a use frequency of the first basic infusion feature satisfies a use-frequency threshold of the enablement criterion.

7. The ambulatory infusion system of claim 1, wherein:
the memory further stores a second basic infusion feature for the ambulatory infusion pump or infusion-management application, wherein the second basic infusion feature is (i) enabled by default and (ii) allows the user to request a bolus of the medicament, wherein the processor is further configured to cause the ambulatory infusion pump to provide the bolus to the user responsive to the user requesting the bolus;
the advanced infusion feature allows the user to indicate a basal rate for the medicament, wherein the processor is further configured to cause the ambulatory infusion pump to provide the medicament to the user at the basal rate responsive to the user indicating the basal rate; and
determining that the enablement criterion is satisfied (i) is based on the competency level of the user and (ii) comprises determining that a number of boluses requested by the user satisfies a bolus-request threshold of the enablement criterion.

8. The ambulatory infusion system of claim 1, wherein:
the processor is further configured to determine the competency level of the user based on two or more of (i) an amount of time since the user activated the ambulatory infusion pump, (ii) an amount of time since the user installed the infusion-management application on the mobile device, (iii) an amount of time since the user paired the ambulatory infusion pump with the mobile device, (iv) a use history of the ambulatory infusion pump or infusion-management application, (v) an amount of time since a diagnosis of the user with the disease, (vi) an age of the user, and (vii) a prescription from a clinician of the user; and
determining whether the enablement criterion is satisfied is based on the competency level of the user.

9. The ambulatory infusion system of claim 1, wherein:
the physiological parameter of the user comprises two or more of (i) a blood glucose level of the user, (ii) a hemoglobin A1C of the user, (iii) an insulin sensitivity of the user, (iv) a heart rate of the user, (v) a blood pressure of the user, and (vi) a body mass index of the user; and
determining whether the enablement criterion is satisfied is based on the physiological parameter of the user.

10. The ambulatory infusion system of claim 1, wherein:
the memory further stores instructions associated with another advanced infusion feature for the ambulatory infusion pump or infusion-management application, wherein the other advanced infusion feature is disabled by default; and
the processor is further configured to:
monitor (i) the competency level of the user or (ii) another physiological parameter of the user;
determine whether another enablement criterion is satisfied based on (i) the competency level of the user or (ii) the other physiological parameter of the user; and
responsive to determining that the other enablement criterion is satisfied, enable the other advanced infusion feature.

11. A mobile device comprising:
an antenna configured to facilitate wireless communication with an ambulatory infusion pump, wherein the ambulatory infusion pump comprises a pump mechanism configured to provide a medicament to a user;
a memory that stores (i) an infusion-management application for managing the ambulatory infusion pump and (ii) an advanced infusion feature for the ambulatory infusion pump or infusion-management application, wherein the advanced infusion feature is disabled by default and ; and
a processor configured to:
execute the infusion-management application;
monitor (i) a competency level of the user with respect to the ambulatory infusion pump, the infusion-management application, or a disease of the user treatable with the medicament or (ii) a physiological parameter of the user;
determine whether an enablement criterion is satisfied based on (i) the competency level of the user or (ii) the physiological parameter of the user; and
responsive to determining that the enablement criterion is satisfied, enable the advanced infusion feature.

12. The mobile device of claim 11, wherein the processor is further configured to:
receive a request for a use of the advanced infusion feature by the infusion-management application;
responsive to receiving the request, determine whether the advanced infusion feature is enabled;
responsive to determining that the advanced infusion feature is enabled, permit the use of the advanced infusion feature; and
responsive to determining that the advanced infusion feature is disabled, deny the use of the advanced infusion feature.

13. The mobile device of claim 11, wherein:
the advanced infusion feature is hidden by default from a user interface of the infusion-management application; and
the processor is further configured to, responsive to determining that the enablement criterion is satisfied, present an indication of the advanced infusion feature via the user interface.

14. The mobile device of claim 13, wherein the processor is further configured to, responsive to determining that the enablement criterion is satisfied, provide a notification to the user via the user interface of the infusion-management application, and the notification indicates the advanced infusion feature is enabled and directs the user to instructions for using the advanced infusion feature.

15. The mobile device of claim 11, wherein the processor is further configured to, after enabling the advanced infusion feature:
monitor (i) interactions of the user with the advanced infusion feature and (ii) a blood-sugar level of the user;
determine whether the advanced infusion feature is negatively impacting the blood-sugar level of the user based on (i) the interactions of the user and (ii) the blood-sugar level of the user; and
responsive to determining that the advanced infusion feature is negatively impacting the blood-sugar level of the user, disable the advanced infusion feature and provide a notification to the user via a user interface of the infusion-management application, wherein the notification indicates the advanced infusion feature is disabled and directs the user to instructions for re-enabling the advanced infusion feature.

16. The mobile device of claim 11, wherein:
the memory further stores a first basic infusion feature for the infusion-management application, wherein the first basic infusion feature is (i) enabled by default and (ii) allows the user to indicate timing of exercises performed by the user, wherein the processor is further configured to adjust operation of the ambulatory infusion pump based on the timing of the exercises;
the advanced infusion feature allows the user to indicate a type or intensity of the exercises, wherein the processor is further configured to adjust operation of the ambulatory infusion pump based on the type or intensity of the exercises; and
determining that the enablement criterion is satisfied (i) is based on the competency level of the user and (ii) comprises determining that a use frequency of the first basic infusion feature satisfies a use-frequency threshold of the enablement criterion.

17. The mobile device of claim 11, wherein:
the memory further stores a second basic infusion feature for the ambulatory infusion pump or infusion-management application, wherein the second basic infusion feature is (i) enabled by default and (ii) allows the user to request a bolus of the medicament, wherein the processor is further configured to cause the ambulatory infusion pump to provide the bolus to the user responsive to the user requesting the bolus;
the advanced infusion feature allows the user to indicate a basal rate for the medicament, wherein the processor is further configured to cause the ambulatory infusion pump to provide the medicament to the user at the basal rate responsive to the user indicating the basal rate; and
determining that the enablement criterion is satisfied (i) is based on the competency level of the user and (ii) comprises determining that a number of boluses requested by the user satisfies a bolus-request threshold of the enablement criterion.

18. The mobile device of claim 11, wherein:
the processor is further configured to determine the competency level of the user based on two or more of (i) an amount of time since the user activated the ambulatory infusion pump, (ii) an amount of time since the user installed the infusion-management application on the mobile device, (iii) an amount of time since the user paired the ambulatory infusion pump with the mobile device, (iv) a use history of the ambulatory infusion pump or infusion-management application, (v) an amount of time since a diagnosis of the user with the disease, (vi) an age of the user, and (vii) a prescription from a clinician of the user; and
determining whether the enablement criterion is satisfied is based on the competency level of the user.

19. The mobile device of claim 11, wherein:
the physiological parameter of the user comprises two or more of (i) a blood glucose level of the user, (ii) a hemoglobin A1C of the user, (iii) an insulin sensitivity of the user, (iv) a heart rate of the user, (v) a blood pressure of the user, and (vi) a body mass index of the user; and
determining whether the enablement criterion is satisfied is based on the physiological parameter of the user.

20. A computer-implemented method for automatic feature management in an ambulatory infusion system, the method comprising:
wirelessly connecting an ambulatory infusion pump and a mobile device, wherein the ambulatory infusion pump comprises a pump mechanism configured to provide a medicament to a user, and the mobile device is configured to execute an infusion-management application for managing the ambulatory infusion pump;
monitoring (i) a competency level of the user with respect to the ambulatory infusion pump, the infusion-management application, or a disease of the user treatable with the medicament or (ii) a physiological parameter of the user;
determining whether an enablement criterion is satisfied based on (i) the competency level of the user or (ii) the physiological parameter of the user; and
responsive to determining that the enablement criterion is satisfied, enabling an advanced infusion feature for the ambulatory infusion pump or infusion-management application, wherein the advanced infusion feature is disabled by default.
